(19) United States
(12) Patent Application Publication
(10) Pub. No.: US 2004/0122498 A1
(43) Pub. Date: Jun. 24, 2004

(54) Pulmonary artery lead for atrial therapy

(76) Inventors: Yongxing Zhang, Little Canada, MN (US); James O. Gilkerson, Stillwater, MN (US); Christopher Paul Knapp, Ham Lake, MN (US)

Correspondence Address: Schwegman, Lundberg, Woessner & Kluth, P.A., P.O. Box 2938, Minneapolis, MN, 55402, US

(21) Appl. No.: 10/325,659

(22) Filed: Dec. 19, 2002

(51) Int. Cl. A61N 1/00

(52) U.S. Cl. 607/122

(57) ABSTRACT

A lead including a lead body configured into a pre-formed J-shape, and a shocking electrode coupled proximate the distal end of the lead body and located distally from a bottom of the pre-formed J-shape, wherein the lead is adapted to be placed within a heart in a J-shaped configuration with the lead extending through the right ventricle and the electrode positioned within a pulmonary artery. The lead can include a distal end adapted for being passively fixated within a pulmonary artery.

### FIELD OF THE INVENTION

This invention relates to the field of medical leads, and more specifically to an implantable lead.

### BACKGROUND

Leads implanted in or about the heart have been used to reverse certain life threatening arrhythmia, or to stimulate contraction of the heart. Electrical energy is applied to the heart via the leads to return the heart to normal rhythm.

For example, one technique to apply a shock to the left atrium of the heart is to implant the electrode through the coronary sinus to reach a location below the left atrium. However, it can be difficult to locate the coronary sinus ostium, thus implantation time can be excessive, or the procedure can be unsuccessful.

### SUMMARY

A lead including a lead body configured into a pre-formed J-shape and a shocking electrode coupled proximate the distal end of the lead body and located distally from a bottom of the pre-formed J-shape. The lead is adapted to be placed within a heart in a J-shaped configuration with the lead extending through the right ventricle and the electrode positioned within a pulmonary artery.

In one aspect, a lead having a lead body extending from a proximal end to a distal end and having an intermediate section, the distal end being adapted for being passively fixated within a pulmonary artery. The lead includes a shocking electrode coupled proximate the distal end of the lead body, wherein the lead is adapted to be placed within a heart in a J-shaped configuration such that the electrode is positioned within the pulmonary artery and the distal end is fixated within the pulmonary artery.

### BRIEF DESCRIPTION OF THE DRAWINGS

FIG. 1 shows a view of a lead, according to one embodiment, implanted within a heart.

FIG. 2 shows a view of a lead, according to one embodiment, implanted within a heart.

FIG. 3 shows a distal portion of a lead according to one embodiment.

FIG. 4A shows a distal portion of a lead according to one embodiment.

FIG. 4B shows a distal portion of a lead according to one embodiment.

FIG. 4C shows a distal portion of a lead according to one embodiment.

FIG. 5 shows a view of a lead, according to one embodiment.

FIG. 6 shows a view of the lead of FIG. 5, implanted within a heart.

FIG. 7 shows a view of a lead, according to one embodiment, implanted within a heart.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

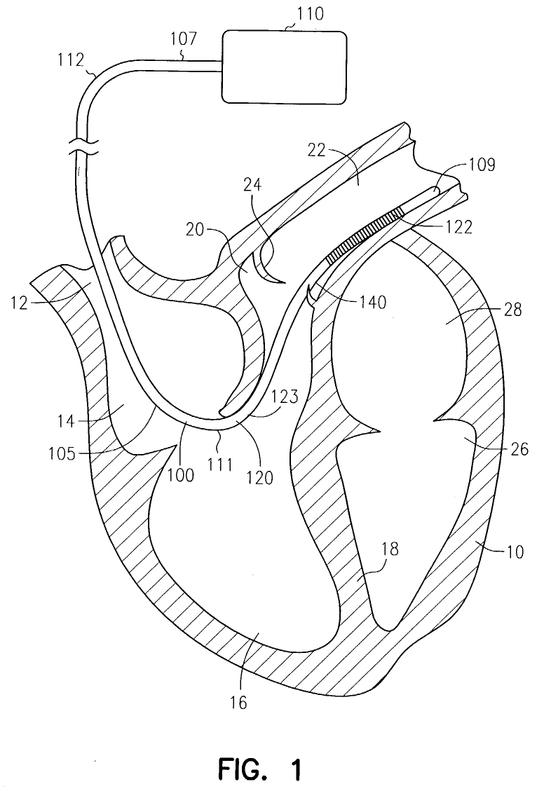

FIG. 1 shows a view of a lead 100 implanted within a heart 10. Heart 10 generally includes a superior vena cava 12, a right atrium 14, a right ventricle 16, a left ventricle 26, a left atrium 28, a ventricular septum 18, and a ventricular outflow tract 20, which leads to a pulmonary artery 22, having a pulmonary artery valve 24. In one embodiment, lead 100 is adapted to deliver defibrillation pulses to heart 10 via an electrode 122. Lead 100 is part of an implantable system including a pulse generator 110, such as a defibrillator.

Pulse generator 110 can be implanted in a surgically-formed pocket in a patient's chest or other desired location. Pulse generator 110 generally includes electronic components to perform signal analysis and processing, and control. Pulse generator 110 can include a power supply such as a battery, a capacitor, and other components housed in a case. The device can include microprocessors to provide processing, evaluation, and to determine and deliver electrical shocks and pulses of different energy levels and timing for ventricular defibrillation, cardioversion, and pacing to heart 10 in response to cardiac arrhythmia including fibrillation, tachycardia, and bradycardia.

In one embodiment, lead 100 includes a lead body 105 extending from a proximal end 107 to a distal end 109 and having an intermediate portion 111. Lead 100 includes one or more conductors, such as coiled conductors, to conduct energy from pulse generator 110 to heart 10, and also to receive signals from the heart. The lead further includes outer insulation 112 to insulate the conductor. The conductors are coupled to one or more electrodes, such as electrode 122. Lead terminal pins are attached to pulse generator 110. The system can include a unipolar system with the case acting as an electrode or a bipolar system.

In one embodiment, electrode 122 includes a shock electrode adapted for delivering shock pulses to heart 10. For instance, lead 100 can be designed for placement of shock electrode 122 within the pulmonary artery 22 to deliver shock pulses to the left atrium 28. In one embodiment, lead 100 is adapted for pulmonary artery placement of shock electrode 122 while utilizing pulmonary artery 22 for lead fixation. For example, in one embodiment electrode 122 is coupled proximate distal end 109. Electrode 122 can have a shocking coil electrode designed to deliver energy pulses of approximately 0.1 to 50 Joules.

In one embodiment, lead body 105 includes a pre-formed, biased J-shape 120 formed in the intermediate portion 111 of the lead body. J-shape 120 is located such that electrode 122 is located distally from a bottom 123 of the pre-formed J-shape 120. Pre-formed J-shape 120 can be in either 2D or 3D. J-shaped portion 120 of lead 100 allows for better placement of electrode 122 within the pulmonary artery. To pre-form the lead, the lead can be manufactured such that it is biased in the J-shape. Thus, the lead naturally reverts to the J-shape when it is implanted. For example, the lead body can be formed in the pre-biased shape or the conductor coils can be formed in the pre-biased shape to bias the lead body into the shape. When implanted, the bottom 123 of the J-shape 120 is within the right ventricle 16 and electrode 122 is positioned past the pulmonary valve 24 such that the electrode is within the pulmonary artery above the left atrium 28.

The pre-formed J-shaped lead design enhances the electrode stability and contact. This can help result in lower thresholds because of better electrode contacts. Moreover, it allows for easier implantation of the lead for delivering pulses to the left atrium. As discussed above, one technique utilizes the coronary sinus to reach the left atrium. This can be a difficult procedure. The present lead and method allow for utilization of the pulmonary artery to deliver the pulses to the left atrium. This allows for shorter and easier implantation techniques.

In one embodiment, at least a portion of lead 100 can include an anti-thrombosis coating 140, such as Hypren or polyethleneglycol for example. Coating 140 can be placed on the lead, for example on the coil electrode or on other segments of the lead.

In some embodiments, lead 100 can be configured to allow both a stylet or catheter delivery. For example, an opening can be left through the middle of the lead to allow a stylet to be used.

In one embodiment, distal end 109 is adapted for being passively fixated within a pulmonary artery. For example, as will be discussed below, a pre-formed biased distal portion 109 can be provided. In some embodiments, to be discussed below, an active fixation technique is utilized. Some embodiments utilize neither passive nor active fixation, relying on the J-shape 120 and gravity to hold the electrode 122 in place within the pulmonary artery.

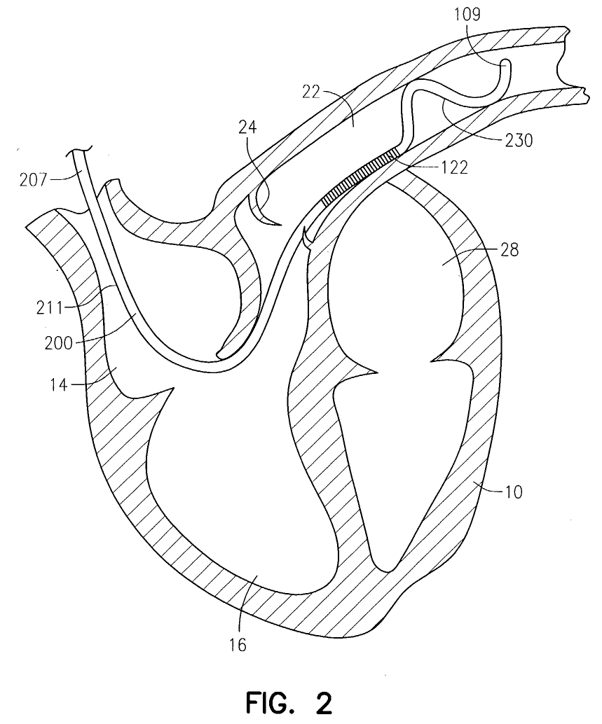

FIG. 2 shows a front view of a lead 200 according to one embodiment, positioned within heart 10. Lead 200 includes some of the components discussed above for lead 100, and the above discussion is incorporated herein. Lead 200 extends from a proximal end 207 to a distal end 209 and includes an intermediate portion 211. Lead 200 can be implanted in heart 10 with distal end 209 located within the pulmonary artery and electrode 122 positioned within the pulmonary artery 22 past valve 24. Some embodiments utilize a branch of the pulmonary artery for fixation of distal end 209.

In one embodiment, lead 200 does not include the pre-formed, biased J-shaped portion 120 discussed above. Lead 200 includes a pre-formed, biased shape 230 on distal end 209 of lead 200. Pre-formed biased shape 230 can include a curved shape such as an S-shape, a C-shape, a J-shape, an O-shape, and other non-linear shapes adapted for contacting one or sides of the pulmonary artery (or a branch of the pulmonary artery) to provide sufficient fixation of the lead. Lead 200 is easier to implant and explant because of the passive fixation which is allowed by shape of distal portion of lead 200. For example, passive fixation allows for easier adjustment of electrode placement, and is easier to explant. Moreover, there is less trauma or perforation to endocardium tissue than with active fixation leads, which can yield lower pacing thresholds. Moreover, there is less trauma to the septal/outflow tract caused by active fixation at the septal/outflow tract location.

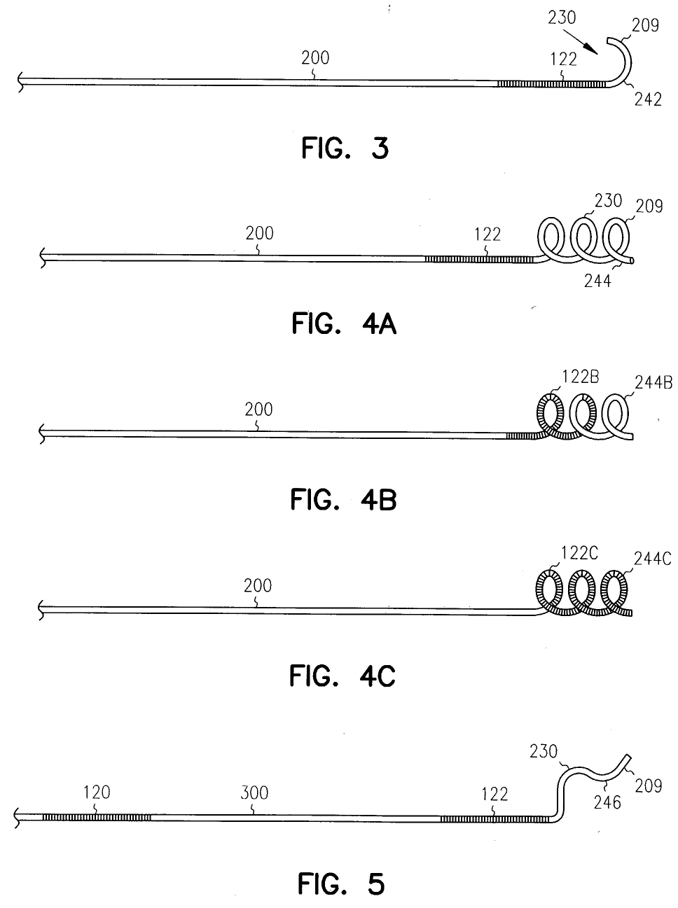

Pre-formed, biased shape 230 can take various configurations. For example, FIG. 3 shows distal portion 209 of lead 200 according to one embodiment. In this example, pre-formed, biased shape 230 includes a J-shaped curve 242 at a distal tip of the lead body. J-shaped curve 242 can be positioned within pulmonary artery 22 or in one of the branch arteries off of the pulmonary artery to fixate the distal end of the lead within the pulmonary artery.

FIG. 4A shows distal portion 209 of lead 200 according to one embodiment. In this example, pre-formed, biased shape 230 includes a spiral configuration 244. The pre-formed, biased shape generally can include at least two lead surfaces which are dimensioned and positionable such that the surfaces contact opposing walls of the pulmonary artery.

The pre-formed biased shapes 230 discussed above and below can also be formed at least partly by the coil electrode itself. For example, FIG. 4B shows lead 200 having a spiral configuration 244B which partially includes a coil electrode 122B formed into a coil shape and at least partially defining spiral configuration 244B. FIG. 4C shows lead 200 having a spiral configuration 244C and a coil electrode 122C covers the distal end of the lead. In these examples of FIGS. 4B and 4C, the coil electrodes 122B and 122C can be pre-formed in the spiral shape to bias the distal end of the lead into the spiral configuration.

FIG. 5 shows a lead 300 according to one embodiment. Lead 300 includes a second electrode 120, such as a coil electrode. In this example, pre-formed, biased shape 230 includes a modified S-shaped configuration 246 to hold the lead within the pulmonary artery or a branch of the pulmonary artery.

In some embodiments, any of the pre-formed biased designs discussed above can also be used on lead 100 having the pre-formed, biased J-shape 120.

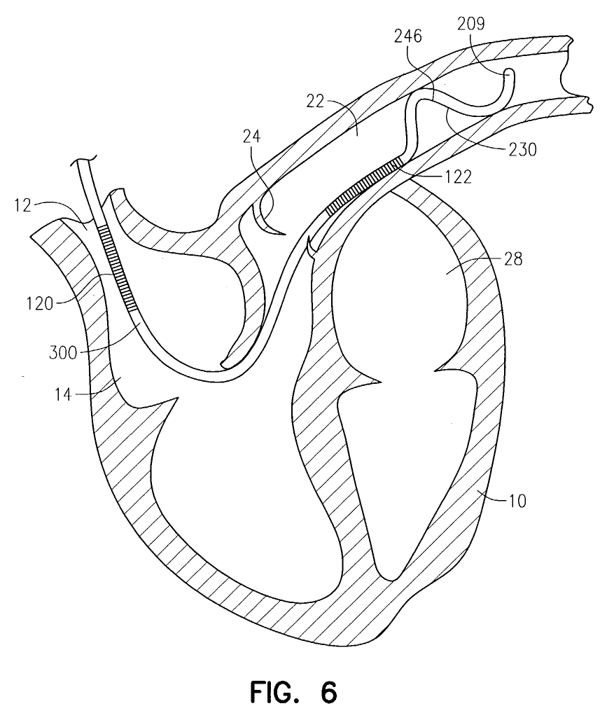

FIG. 6 shows lead 300 implanted within heart 10 such that electrode 120 is within the superior vena cava 12 or right atrium 14, and electrode 122 is within pulmonary artery 22, past valve 24. In use, a therapy system utilizing lead 300 can deliver shocks for left atrial defibrillation, right atrial defibrillation, biatrial defibrillation, or be used as a triad system using the pulse generator case as an electrode. In some embodiments, lead 300 can include a pre-formed J-shape such as shape 120 discussed above.

In one example use of one or more of the leads discussed herein, the lead is inserted through the right ventricle and into the pulmonary artery using a guiding catheter or a stylet. The lead is positioned until the distal end of the lead is within the pulmonary artery and a shock electrode is located past the pulmonary valve. In one embodiment, the lead can be held in place by either the pre-formed J-shape or the passive fixation techniques discussed above. The lead is coupled to a pulse generator, and when the pulse generator detects a need for therapy, the shock pulse is delivered via electrode 122 in either a bipolar or unipolar system. For example, the pulse generator can deliver energy pulses of approximately 0.1 to 50 Joules via the electrode to the left atrium. In some embodiments, a second electrode can be provided for location within the superior vena cava, as discussed above.

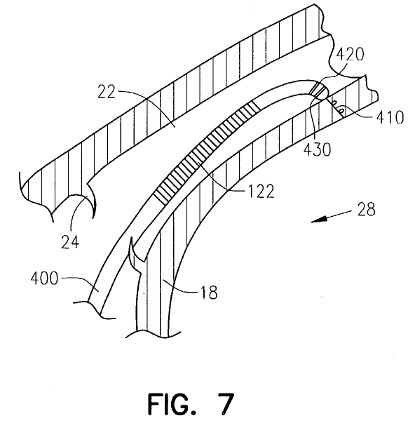

FIG. 7 shows a view of a lead 400 according to one embodiment, implanted within a heart 10. Lead 400 can include one or more of the components discussed above for leads 100 and 200 and 300 and the above discussions are incorporated herein. In one embodiment, lead 400 is adapted to be actively fixated within the pulmonary artery 22 utilizing a helix 410 or other fixation mechanism, for example. Lead 400 includes electrode 122 which is positionable to apply energy pulses to left atrium 28. In one embodiment, lead 400 includes radiopaque markers 420 near the distal tip to help a physician guide the lead when viewed under fluoroscopy. One embodiment includes a drug elution member 430, which can elute steroids, for example, to reduce inflammatory response of the tissue. In some embodiments, active fixation can be provided in addition to or in place of the passive fixation design discussed above.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A lead comprising:
a lead body extending from a proximal end to a distal end and having an intermediate portion, wherein the lead body is configured into a pre-formed J-shape; and
a shocking electrode coupled proximate the distal end of the lead body and located distally from a bottom of the pre-formed J-shape, wherein the lead is adapted to be placed within a heart in a J-shaped configuration with the lead extending through the right ventricle and the electrode positioned within a pulmonary artery.

2. The lead of claim 1, wherein the distal end of the lead is adapted to be passively fixated within the pulmonary artery.

3. The lead of claim 2, wherein the distal end of the lead includes a pre-formed biased shape.

4. The lead of claim 1, wherein the lead is adapted to be positioned such that the a shock can be delivered to the left atrium via the electrode.

5. The lead of claim 1, wherein at least a portion of the lead includes an anti-thrombus coating.

6. A lead comprising:
a lead body extending from a proximal end to a distal end and having an intermediate section, the distal end being adapted for being fixated within a pulmonary artery; and
a shocking electrode mounted proximate the distal end of the lead body;
wherein the lead body includes a pre-formed J-shape along the intermediate section of the lead body with a base of the J-shaped lead body being proximally located relative to the electrode such that the electrode is past a pulmonary artery valve when the lead is positioned in a heart with the intermediate section within the right ventricle and the distal end within a pulmonary artery.

7. The lead of claim 6, wherein the distal end of the lead includes a biased pre-formed shape adapted for passively fixating the distal end within the pulmonary artery.

8. The lead of claim 7, wherein the pre-formed, biased shape includes an S-shaped configuration.

9. The lead of claim 7, wherein the pre-formed, biased shape includes a spiral configuration.

10. The lead of claim 7, wherein the pre-formed, biased shape includes a J-shaped curve at a distal tip of the lead body.

11. The lead of claim 7, wherein the pre-formed, biased shape includes at least two surfaces positioned to contact opposing walls of the pulmonary artery.

12. The lead of claim 7, wherein the pre-formed, biased shape includes a shape adapted to fixate the distal end within a branch of the pulmonary artery.

13. The lead of claim 6, wherein at least a portion of the lead includes an anti-thrombosis coating.

14. A lead comprising:
a lead body extending from a proximal end to a distal end and having an intermediate section, the distal end being adapted for being passively fixated within a pulmonary artery; and
a shocking electrode coupled proximate the distal end of the lead body, wherein the lead is adapted to be placed within a heart in a J-shaped configuration such that the electrode is positioned within the pulmonary artery.

15. The lead of claim 14, wherein the distal end of the lead includes a biased pre-formed shape adapted for passively fixating the distal end within the pulmonary artery.

16. The lead of claim 15, wherein the pre-formed, biased shape includes an S-shaped configuration.

17. The lead of claim 15, wherein the pre-formed, biased shape includes a spiral configuration.

18. The lead of claim 15, wherein the pre-formed, biased shape includes a J-shaped curve at a distal tip of the lead body.

19. The lead of claim 15, wherein the pre-formed, biased shape includes at least two surfaces positioned to contact opposing walls of the pulmonary artery.

20. The lead of claim 15, wherein the pre-formed, biased shape includes a shape adapted to fixate the distal end within a branch of the pulmonary artery.

21. The lead of claim 14, wherein at least a portion of the lead includes an anti-thrombus coating.

22. The lead of claim 14, further comprising a second shocking electrode coupled to the lead and adapted to be positioned in a superior vena cava or a right atrium.

23. The lead of claim 14, wherein the electrode is adapted to deliver shocks of energy ranging from approximately 0.1 Joules to approximately 50 Joules.

24. A method comprising:
providing a lead having a lead body extending from a proximal end to a distal end and having an intermediate portion, the lead having a shocking electrode coupled proximate the distal end, wherein the lead body includes a pre-formed J-shape along the intermediate portion; and
inserting the lead through a right ventricle and into a pulmonary artery such that the shocking electrode is within the pulmonary artery.

25. The method of claim 24, wherein the distal end of the lead includes a pre-formed, biased shape adapted to passively fixate the distal end of the lead within a pulmonary artery.

26. The method of claim 24, further comprising delivering pulses from the electrode to a left atrium.

27. A method comprising:
providing a lead having a lead body extending from a proximal end to a distal end and having an intermediate portion, the lead having a shocking electrode coupled proximate the distal end, wherein the distal end of the lead includes a pre-formed, biased shape adapted to passively fixate the distal end of the lead within a pulmonary artery; and
inserting the lead through a right ventricle and into a pulmonary artery such that the shocking electrode is within the pulmonary artery and the distal end is fixated within the pulmonary artery.

28. The method of claim 27, wherein the pre-formed, biased shape includes an S-shaped configuration.

29. The method of claim 27, wherein the pre-formed, biased shape includes a spiral configuration.

30. The method of claim 27, wherein the pre-formed, biased shape includes a J-shaped curve at a distal tip of the lead body.

31. A lead comprising:
a lead body extending from a proximal end to a distal end; and
a shocking electrode coupled proximate the distal end of the lead body, wherein the lead is adapted to be placed within a heart in a J-shaped configuration such that the electrode is positioned within the pulmonary artery;
wherein the distal end of the lead is adapted to be actively fixated within the pulmonary artery.

32. The lead of claim 31, wherein the lead includes a radiopaque marker proximate the distal end.

33. The lead of claim 31, wherein the lead includes a drug elution member proximate the distal end.
